(19) United States
(12) Patent Application Publication
(10) Pub. No.: US 2024/0366912 A1
(43) Pub. Date: Nov. 7, 2024

(54) INTERACTIVE RESPIRATORY RELAXATION DEVICE AND METHODS THEREOF

(71) Applicant: Erella PINES, Pardes Hanna (IL)

(72) Inventor: Erella PINES, Pardes Hanna (IL)

(21) Appl. No.: 18/689,069

(22) PCT Filed: Sep. 14, 2022
(86) PCT No.: PCT/IL2022/050996

§ 371 Date: Mar. 4, 2024

Related U.S. Application Data
(60) Provisional application No. 63/243,902, filed on Sep. 14, 2021.

(51) Int. Cl. A61M 21/02; A61M 21/00; G16H 40/63

(52) U.S. Cl. CPC A61M 21/02; G16H 40/63; A61M 2021/0022; A61M 2205/10; A61M 2205/18; A61M 2205/3303; A61M 2205/3327; A61M 2205/3331; A61M 2205/3553; A61M 2205/3561; A61M 2205/3592; A61M 2205/505; A61M 2205/581; A61M 2205/582; A61M 2205/583; A61M 2205/8206; A61M 2209/088; A61M 2230/40

(57) ABSTRACT

An interactive respiratory relaxation auto-biofeedback device for relieving stress characterized by at least three continuously-intercommunicated modules: at least one bladder and at least one pressure sensor in connection with the at least one bladder; the at least one bladder configured for manual actuation by a user during exhalation; at least one programmable actuator for inflating and deflating the at least one bladder; an actuating module, comprising computerized feedbacking module (CFM) for programming and controlling the programmable actuator of the at least one bladder, and a self-reporting switch for user fixing of the breathing rate set by the actuating module.

### FIELD OF THE INVENTION

The present invention pertains to an interactive and active respiratory guidance relaxation and stress-relief device and to methods thereof.

### BACKGROUND OF THE INVENTION

This invention is to provide a respiratory relaxation guiding device and device that leads the user to effective and rapid relaxation through a sequence of breathing cycles, and increase the time of the breathing cycle as it goes along, without the need for input data, learned skills or practicing.

To date, a large part of the population remains unfamiliar with relaxation practices such as meditation and guided imagery. Thus, when stressed, many people do not know what to do, nor have the time required to practice relaxation properly, should they be familiar with it.

Voluntary breathing control is probably one of the oldest-known stress-management technique. It is effective both in alleviating stress levels and in helping to relax muscles and joints made tense by anxiety or worry.

When children or adults feel stressed out, afraid, scared, uneasy and lack concentration-breathing becomes more rapid and shallow. The initial phase and currently known technique designed to reduce stress and anxiety is voluntary slowing of breathing rate.

Voluntary slow breathing is a proven method to alleviate medical problems brought on by stress and anxiety and to prevent the development of medical conditions affecting normal functioning.

Children and adults encounter during their lifetime health conditions (diseases, invasive tests and treatments, and others) that cause them immediately restlessness, lack of concentration and anxiety.

A known medical phenomenon is that anxiety and stress slow the ability to heal illnesses and the recovery from surgeries and injuries. Also, children and adults that are exposed to stress in their lives may further suffer of insomnia, psychotic diseases, irritability and lack of concentration. Various chronic diseases like diabetes, asthma, autoimmune diseases and other malignancies can bring the patients short-or long-term anxiety.

Invasive treatments in child and adult are sources of anxiety. These treatments can be non-recurring treatments such as X-rays, blood tests, surgery or prolonged treatments such as dental care, chemical treatments, radiation and others. This anxiety occurrences may occur at any time before, during or after treatment.

Other states of anxiety are found in the education system, like kindergardens, schools and universities, in the army during training or during war, in prisons, in a variety of social services and in the general public in times of crisis.

Furthermore, there are people who want to enter into a state of relaxation without the need of a coach or a doctor.

Certain relaxation and stress-relief methods, such as meditation, put the person in a relaxed mode which reduces the levels of anxiety. Yet this type of relaxation is a learned skill. It must be practiced with a qualified instructor either individually or in small groups.

In the art there are several inventions related to this issue. In general, these inventions disclose devices that indicate to a user if he is in stress, how much in stress is the user, the timing of the next breath, either as the beginning of an inhalation, the beginning of an exhalation, or both. In many of the devices, the device measures at least one of the user's heart rate, pulse rate and breathing rate and, from the measured data and other, stored, data, calculates the timing of the beginning of inhalation and/or exhalation.

The means of indication to the user can be visual (such as a light, a symbol on a display screen, or a graphic display), audible (such as a beep, or a piece of music), tactile (such as a vibration or other pressure on the user) or any combination of these.

It was shown before that a tactile interface of a breathing assistance device is provided useful by using a shape-changing airbag. For most participants, the overall heart rate variability is improved after breathing training. Moreover, “Breathe with Touch” brought users better satisfaction during the exercise, see Yu, Bin, et al. “Breathe with touch: a tactile interface for breathing assistance device.” IFIP Conference on Human-Computer Interaction. Springer, Cham, 2015. Nevertheless, this “Breathe with touch” mechanism routinely oscillates in a non-feedbacked manner between an inflate configuration and a deflate configuration, in such a manner that the user is very much passive when follows said shape-changing. A device and a method which ensure compliance of the user with the inflating/deflating moment is still a long-felt need.

Even though there have been extensive solutions offered, they are all inadequate and there is still a long felt need for a device that is easy to use, is interactive, and comprises multifunctional capabilities.

### SUMMARY OF THE INVENTION

The present invention discloses an interactive device which detects, analyzes and correlates data from multiple sources: the user's objective physiological measurements (biofeedback), the user's subjective input (auto-biofeedback), and external database resourse/s. The device is thus aimed to regulate the breath rate of the user and act as his/her unanimated relaxation coach.

It is hence one object of the invention to disclose an interactive respiratory relaxation auto-biofeedback device for relieving stress characterized by at least three continuously-intercommunicated modules: at least one bladder and at least one pressure sensor in connection with the at least one bladder; the at least one bladder configured for manual actuation by a user during exhalation; at least one programmable actuator for inflating and deflating the at least one bladder; an actuating module, comprising computerized feedbacking module (CFM) for programming and controlling the programmable actuator of the at least one bladder, and a self reporting switch for user fixing of the breathing rate set by said actuating module.

It is another object of the invention to disclose the device as defined above, wherein the device further comprises at least one wearable sensor for receiving and transmitting breathing data; and a processor for receiving the breathing data, processing the received breathing data, and sending instructions to the actuator for inflating and deflating the at least one bladder.

It is another object of the invention to disclose the device as defined in any of the above, wherein the at least one actuator is selected from the group consisting of: a valve, a pump, a spring, and any combination thereof.

It is another object of the invention to disclose the device as defined in any of the above, wherein the at least one wearable sensor is configured for sensing regular breathing or diaphragmatic breathing.

It is another object of the invention to disclose the device as defined in any of the above, wherein the wearable sensor comprises at least one accelerometer configured to measure the movement of a body part to which the at least one wearable sensor is attached.

It is another object of the invention to disclose the device as defined in any of the above, wherein the at least one wearable sensor has a noise filtering mechanism.

It is another object of the invention to disclose the device as defined in any of the above, wherein the actuating module is operable in a method comprising steps: by means of the CFM, directing a user for breathing and actuating the at least one bladder in a defined rate, namely a rhythm B_{I/D}R to both (i) breathing in a hereto defined rate, namely inhaling/exhaling in the directed rhythm (U_{I/E}R), and concurrently (ii) inflating/deflating the at least one bladder in the hereto defined rhythm (B_{I/D}R); and further concurrently, releasing excess air via the valve from the at least one bladder in a rhythm (U_{P/R}R); and further concurrently, by means of the at least one wearable sensor, sensing the user for a breathing rhythm (U_{I/E}R).

It is another object of the invention to disclose the device as defined in any of the above, wherein if user's breathing rhythm U_{I/E}R correlates with the CFM indicated B_{I/D}R, indicating B_{I/D}R until first members of a group consisting following steps is provided: (i) user indicating “I Feel Good”; (ii) CFM indicating program is automatically terminated; and (iii) user's breathing rhythm U_{I/E}R does not corrollate any longer with the CFM indicated B_{I/D}R, resulting with said CFM indicating end of procedure.

It is another object of the invention to disclose the device as defined in any of the above, wherein if user's indicating “I Feel Good” and user's UI/ER correlates with BI/DR for a predefined period of time, the CFM indicates end of procedure.

It is another object of the invention to disclose the device as defined in any of the above, the programmable actuator comprises a termination program, wherein if the termination program is activated, the CFM indicating end of procedure.

It is another object of the invention to disclose the device as defined in any of the above, wherein if user's breathing rhythm U_{I/E}R does not correlate with the at least one bladder's actuating rhythm B_{I/D}R for more than a predefined period of time, further providing steps being members of a group consisting of (i) indicating the user (e.g., alerting by means of lights, sounds, vibration, reminding by audio or text messages), by means of the CFM, to further pressing/releasing the at least one bladder in the indicated rhythm B_{I/D}R; (ii) alerting the user or medical personnel in close or remote location; and (iii) altering rhythm B_{I/D}R, resulting with said CFM indicating end of procedure.

It is another object of the invention to disclose the device as defined in any of the above, wherein the at least one bladder is passive, in that it does not deflate until a user presses the at least one bladder.

It is another object of the invention to disclose the device as defined in any of the above, further characterized by the device being connected to at least one additional device, and being configured to upload or download data to/from the at least one additional device.

It is another object of the invention to disclose the device as defined in any of the above, wherein the computerized feedback module (CFM) is a mobile device.

It is another object of the invention to disclose the device as defined in any of the above, further comprising a user interface (UI).

It is another object of the invention to disclose the device as defined in any of the above, wherein the user interface (UI) is a mobile device.

It is another object of the invention to disclose an interactive respiratory relaxation auto-biofeedback method for relieving stress characterized by at least three continuously-intercommunicating modules, the method comprising steps of: obtaining at least one bladder and at least one pressure sensor in connection with the at least one bladder; the at least one bladder configured for manual actuation by a user during exhalation; and operating the at least one bladder; obtaining at least one programmable actuator for inflating and deflating the at least one bladder; and operating the at least one programmable actuator; obtaining an actuation module, comprising computerized feedbacking module (CFM) for programming and controlling the actuation of the at least one bladder, and a self reporting switch for user fixing of the breathing rate set by the actuating module; and operating the actuation module.

It is another object of the invention to disclose the interactive respiratory relaxation auto-biofeedback method as defined above, wherein the method further comprises steps of: obtaining at least one wearable sensor for receiving and transmitting breathing data; and operating the at least one wearable sensor; obtaining a processor for receiving the breathing data, processing the received breathing data, and sending instructions to the actuator for inflating and deflating the at least one bladder; and operating the processor.

### BRIEF DESCRIPTION OF THE DRAWINGS

FIG. 1 shows a schematic representation of some of the parts belonging to the device of the present invention;

FIG. 2a-e shows schematic representations of other embodiments of the device of the present invention;

FIGS. 3a-e shows schematic representations of different embodiments of the present invention;

FIG. 4 shows a graphical representation of one embodiment of the present invention;

FIG. 5 illustrates CIRRB device of one embodiment of the present invention; and

FIG. 6a-b shows schematic representations of additional embodiments of the device of the present invention.

### DETAIL DESCRIPTION OF THE PREFERRED EMBODIMENTS

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions. It is understood that the phraseology and terminology employed herein is not to be construed as limiting and are for descriptive purpose only.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques and procedures either known to, or readily developed from known manners, means, techniques and procedures by practitioners of the art to which the invention belongs. The descriptions, examples, methods and materials presented in the claims and the specification are not to be construed as limiting but rather as illustrative only.

Meanings of technical and scientific terms used herein are to be commonly understood as to which the invention belongs, unless otherwise defined. The present invention can be implemented in the testing or practice with methods and materials equivalent or similar to those described herein.

As used herein, the singular form “a”, “an” and “the” include plural references, unless the context clearly dictates otherwise. For example, the term “a compound” or “at least one compound” may include a plurality of compounds, including mixtures thereof. The word “optionally” is used herein to mean “is provided in some embodiments and not provided in other embodiments”. Any particular embodiment of the invention may include a plurality of “optional” features unless such features conflict.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the following claims.

This invention is to provide a respiratory relaxation guiding device and device that leads the user to interactive effective and immediate relaxation threw a sequence of breathing cycles, and increase the time of the breathing cycle as it goes along, without the need for learned skills or practicing.

Certain relaxation methods, such as meditation, put the person in a relaxed mode which reduces the levels of anxiety. Yet this type of relaxation is a learned skill. It must be practiced with a qualified instructor either individually or in small groups.

To date, a large part of the population remains unfamiliar with these relaxation practices. Thus, when stressed, many people do not know what to do, nor have the time required to practice relaxation properly, should they be familiar with it.

Voluntary breathing control is probably the oldest-known stress-management technique. It is effective both in alleviating stress levels and in helping to relax muscles and joints made tense by anxiety or worry.

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a Device for Active Breathing Assistance (DABA) adapted to provide an interactive respiratory relaxation and stress-relief device, software and method.

The term “Device for Active Breathing Assistance (DABA)” refers hereinafter to the device of the present invention.

The term “external device” refers hereinafter to the secondary devices where the software is installed.

The term “User(s)” refers hereinafter to any able-users or people with the cognitive and physical abilities to synchronize their breathing with the movements and stimuli emitted by DABA.

The term “wearable” refers hereinafter to wearable technology (also called wearable gadgets) is a category of technological devices that can be worn by a consumer and often include tracking information related to health and fitness. Other wearable tech gadgets include devices that have small motion sensors to take photos and sync with your mobile devices.

The term “motor” refers hereinafter to any class of electrical machines that converts direct current electrical power into mechanical power.

The term “LED” refers hereinafter to any class of light-emitting diode, or any kind of light that can be used to signal the user.

The term “biofeedback” refers hereinafter to any class of the process of gaining greater awareness of many objectively measurable physiological functions of he user's body, by using electronic or other instruments, and with a goal of being able to manipulate the body's devices at will.

The term “auto-biofeedback” refers hereinafter to any class of the process of gaining greater awareness of any subjectively identified physiological functions of the user's body, by using any appropriate binary or spectral evaluation of sensation, including but not limited to self-assessed scales of pain (such as Wong-Baker FACES Pain Rating Scale; Coloured Analogue Scale; Visual Analog Scale (VAS); Verbal Numerical Rating Scale (VNRS); Verbal Descriptor Scale (VDS); Brief Pain Inventory, etc.), anxiety (such as Hamilton Anxiety Rating Scale (HAM-A), Beck Anxiety Inventory (BAI), etc.), psychological stress (such as Holmes and Rahe stress scale, etc.), phychological trauma (such as SOTS, etc.), a user-generated statement indication such as “I feel good”, etc.

The term “wearable sensor” refers hereinafter to any class of sensors attached to a wearable sensor module, measuring accerleration of movement of any relevant body part as a tool of measuring user's breathing.

The term “noise filtering mechanism” refers hereinafter to any class of mechanisms configured to recognize and filter out noise input indicated by a device, such as artifactual acceleration measured by an accelerometer.

The term “processor” refers hereinafter to any class of private contents (be it electrical, mechanical or biological) which takes information (a sequence of enumerated symbols or states) in one form and processes (transforms) it into another form, e.g. to statistics, by an algorithmic process.

The term “actuating module” refers hereinafter to any class of component of a machine or a device that is responsible for moving and controlling a mechanism or device, for example by opening/closing a valve or activating/deactivating a pump.

The term “diaphragmatic breathing” refers hereinafter to any class of breathing that is done by contracting the diaphragm, a muscle located horizontally between the thoracic cavity and abdominal cavity. Air enters the lungs as the diaphragm strongly contracts, but unlike during traditional relaxed breathing (eupnea) the intercostal muscles of the chest do minimal work in this process. The belly also expands during this type of breathing to make room for the contraction of the diaphragm.

The term “regular breathing” refers hereinafter to any class of traditional relaxed breathing (eupnea) that is not diaphragmatic breathing.

It is the intention of the present invention to provide a Device for Active Breathing Assistance (DABA) that is an interactive connected device allowing any users, to discover, test, practice, and “psychologically and physically” enjoy the scientifically and medically proven health benefits of deep-breathing and focused breathing.

The present invention can also be used by professionals and their respective users (clients, patients) to learn, to teach, to practice, specific breathing patterns that can improve the quality of their performances in their relevant field of activities, such as: speech correction, singing, running, stress release, anxiety management and emotional control.

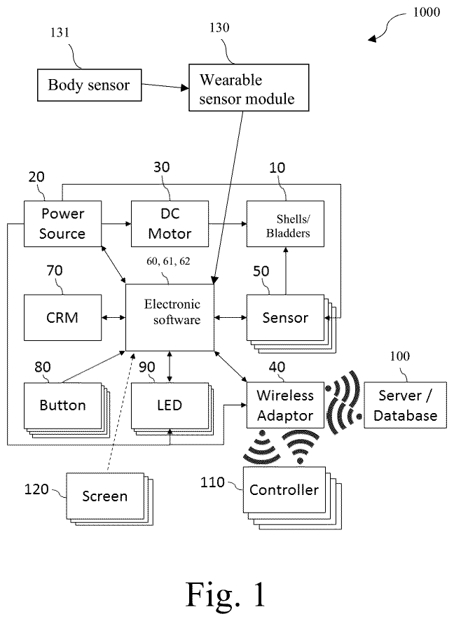

Referring now to FIG. 1, showing a graphical representation of one embodiment of the present invention. In said embodiment of the present invention 1000, DABA comprise two (2) shells (“wings”) or one or more of inflatable/deflatable bladders 10, power source 20, Motor 30, wireless adaptor 40, pressure sensor(s) 50, electronic integrated micro-controller 60, internal software 61, UX/UI software 62, a flash memory or any other computer readable medium (CRM) 70, buttons 80, LEDs 90, optional screens, database 100, data servers 100, wearable sensor module 130, and one or more wearable sensors 131.

DABA's shells (“wings”) 10 can be made of any material such as plastic, metals, wood and others. The shells' function is to contain, protect and provide a satisfactory experience to the user. Therefore, the shell is customable to the touch preferences of the user.

In other embodiments, there are one or more of inflatable/deflatable bladders 10 instead of shells. These bladder can be made of any material such as plastic, rubber or sponge. The bladders' function is to contain, protect and provide a satisfactory experience to the user. Therefore, the bladders are customable to the touch preferences of the user.

ABA's source of power 20 can be a rechargeable battery or a replaceable battery. The source of power provide power to the micro-controller 60, to the LEDs 90 and to the Motor 30 when in use.

DABA's Motor 30, is directly connected to the shells 10 and can move them in both directions, from closed position to the maximum opened angle, when the integrated controller gives the command.

DABA's wireless adaptor 40, synchronize and transfer data to the external device 110, when DABA is “ON” and connected to the external device 110.

DABA's pressure sensor(s) 50, comprise: a. a Motor Strain Sensor, to detect if the user is applying force to open or close the DABA; b. Wings Contact Sensors, to indicate that the user is holding the DABA as recommended.

DABA's electronic integrated micro-controller 60, is responsible for controlling and coordinating between all the parts of the device. The micro-controller 60 is connected to the power source 20 which activates the Motor 30 that move the shells 10. The micro-controller 60 receives the necessary energy from the power source 20. The sensors 50 transmit the information received to the micro-controller 60 which, based on the software, may activate the shells 10, the LEDs 90 and further transmit said information to the external device 110 or the databases in the servers 100 via the wireless adaptor 40.

DABA's internal software 61 and wireless adaptor 40, enable synchronization with the external device 110, enables control of the Motor 30 and the LEDs 90 and enables collection of data from the sensors 50.

DABA's UX/UI software 62 (App, Web-App, Desktop-App), is to help the user to use the DABA, access to the database 100 and services online as well as manage the private information of his client account.

DABA's flash memory or CRM 70, enables to store the data to operate the DABA and the data collected from the DABA.

DABA's buttons 80, actuate several functions: Switch #01—“on”, when the user opens the DABA, the DABA is turned on; Switch #02—“Keep the speed”, when the user presses it, it locks the DABA movements on the current frequency; Switch #02—“Control the speed”, user can increase or decrease the frequency of the active breathing pattern; Switch #02—“Stop”, long press on the button turns the DABA off; Switch #03—“Feel Good button”, the user can indicate during or after a session his positive feelings by pressing the button; Switch #04—“Choose Mode”, the user can choose the mode he wants to activate by pressing on the button to cycle between the modes programmed into the DABA. Default position is mode 1; and Switch #05—“Start button”, the user press it when ready to start a breathing session.

DABA's LEDs 90, are used to provide visual signals to the user. DABA's wearable sensor module 130 is connected to one or more wearable sensors 131. These sensors measure are configured for sensing user's regular breathing or diaphragmatic breathing by utilizing accelerometers to measure the movement of a body part to which said sensor is attached (usually chest or abdomen), and the direction of said a in relation to the magnetic field of Earth, and by utilizing a mechanism to filter out noise.

DABA may also comprise optional screens 120, which provide visual information to the user. In certain embodiments the DABA can be controlled via a touch-screen.

DABA can connect to several databases 100, via the wireless adaptor 40. All the information regarding internal software, updates, software for “controllers” reside in these databases. All the databases are allocated in specialized data servers 100. Also, personal and behavioral information of users are saved in these databases.

The device can further include a feedback mechanism such as a thimble which is placed on the user's finger or palm when the hand/s are holding the DABA. The thimble checks stress conditions (such as galvanic skin response or other parameters) and translates it to output that appears on a display. The output can be in various forms such as a graph, changes in light intensity or others.

Preferably, the device further includes a set of ear phones that connect to the DABA and/or external device 110 and enable the user to listen to a choice of music or rhythms that can be optionally and automatically (typically, via software control) synchronized with the breathing rate.

In another embodiment the opening and closing movements created by the Motor 30 can be replaced by spring(s) that perform the same function or indeed any other physical mechanism that allows the same operative mode of induced tactile cyclical activity.

In another embodiment the device can be configured as a wearable computerized device.

In one embodiment DABA is a stand-alone product that can carry several pre-programmed modes. In addition, when DABA is connected to the external device 110, the user can choose additional programs and content such as media functions, videos, sounds and music.

Furthermore, DABA can be connected to a software that can be installed on smartphones, PC, Laptops, tablets (external device 110), using a wireless connection such as Bluetooth or a physical connection via micro-USB.

The aforementioned software will be compatible for most Operating Devices, Apple, PC and mobile devices, Windows, IOS, Android, Applications, Web-App (Web based application via internet browsers) and Desktop Apps.

In another embodiment the user is free to hold the DABA in a way that is comfortable for him/her: one hand, 2 hands, only with the fingers (see mode 3 bellow for specific recommended postures), between the legs, the head or any other part of the body of the user. This is crucial to emphasize the user experience and to give a sense of embracing and focus, which helps the user to relax.

In another embodiment DABA can be taken everywhere thanks to its small size.

It should be clear that the examples provided in the description are in no-way intend to limit the present invention. Several configurations can be made in order to achieve the same result: a device for active breathing assistance that can be used with any part of the body of the user, can be wearable and configurable for multiple methodologies.

As a manner of example, instead of using two shells (or wings), the interactive device can be bladders that inflate and deflate at a certain rhythm.

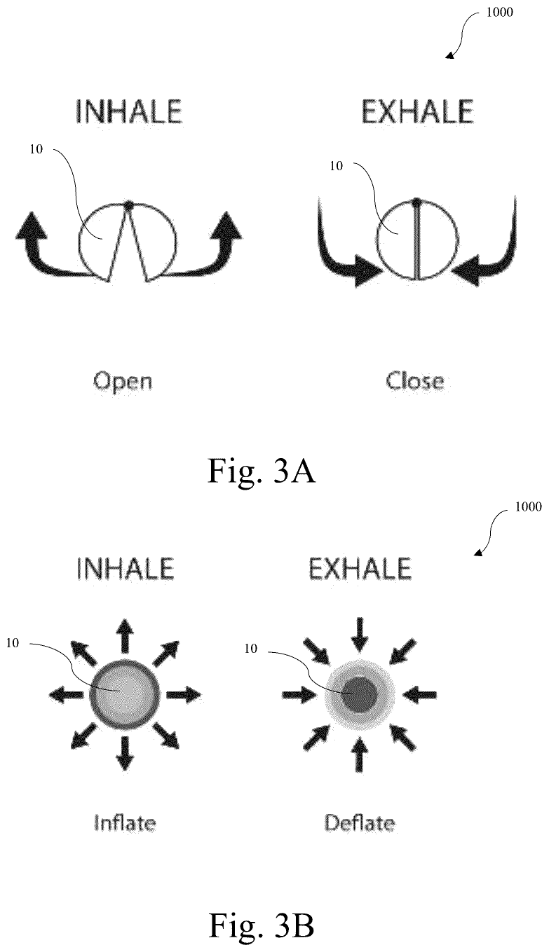

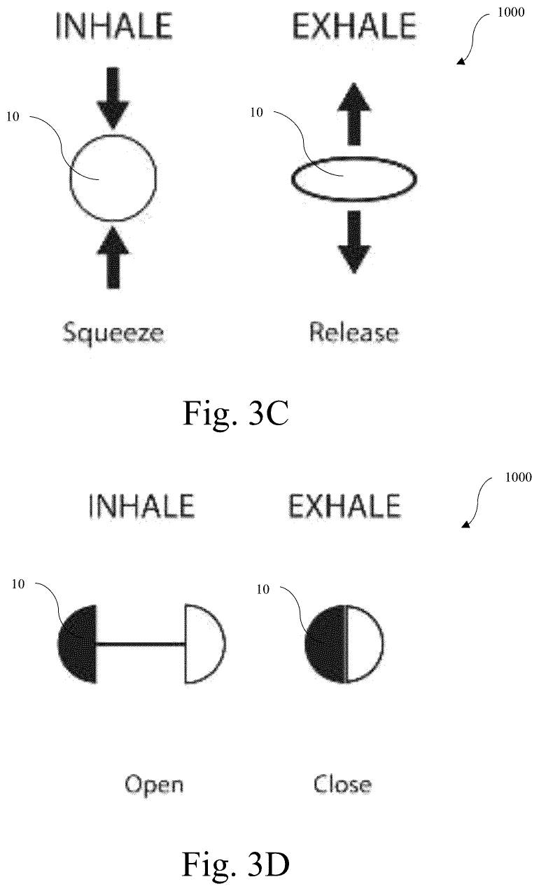

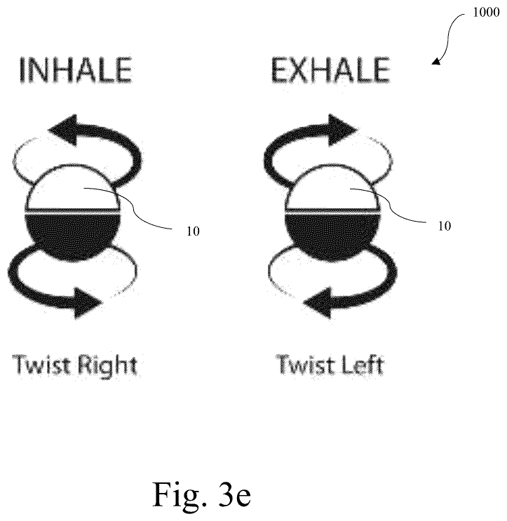

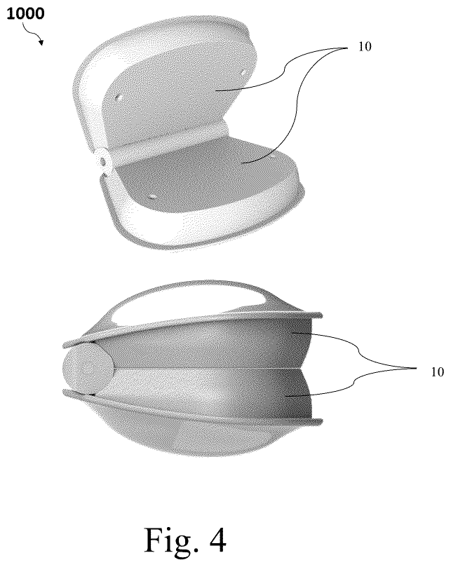

Additional embodiments comprising two shells (or wings) are presented in figures FIG. 3A, 3D, FIG. 3E and FIG. 4.

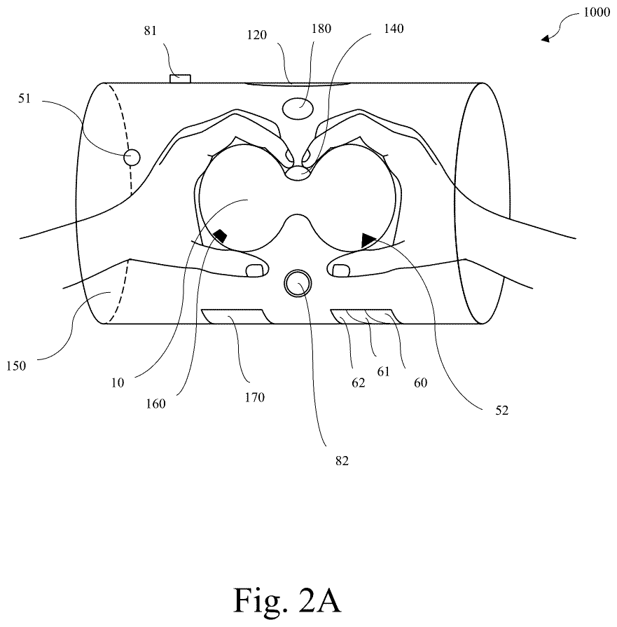

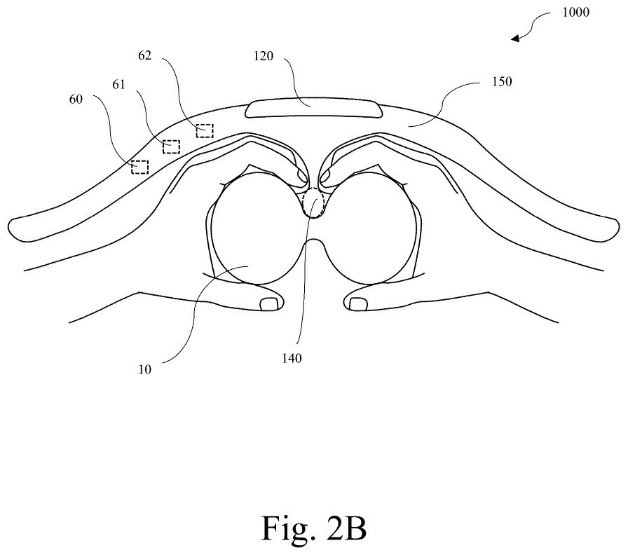

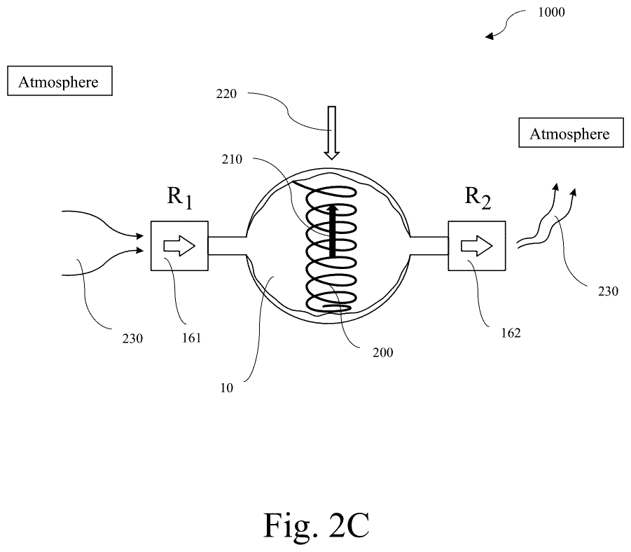

Referring now to FIG. 2A, showing a schematic device of another embodiments of the present invention 1000. The device is formed by a sleeve 150 in which the user inserts both hands. Inside the sleeve there is a dual bladder 10 so the user may grab each side of the bladder with each hand. The bladder is connected to a bidirectional pump 170 enabled to insert and remove air from the bladder 10-thereby inflating or deflating said bladder 10. The pump 170 is controlled by an electronic circuit which comprises all the required parts to operate the device (hardware and software): internal software 61, UX/UI software 62, and electronic integrated micro-controller 60. The sleeve 150 also comprises a screen 120 that helps interaction between the device and the user. A switch 81 is configured to turn on or off the device. The bladders 10 comprise a pressure sensor 52. Said pressure sensor 52 senses when the user applies force to the bladders 10 and transmits the information to the electronic circuit which compares between the movements of the user and the executed program in question. The bladders also comprise a release valve 160 that enables the bladders 10 to be deflated rapidly and efficiently.

In another embodiment of the invention a continuously interactive respiratory relaxing biofeedback device (CIRRB), modules, mechanism and methods for continuously bio-feedbacked relaxation is disclosed.

An interactive respiratory relaxing auto-biofeedback device for relieving stress is hereto described and illustrated. The CIRRB device is characterized by four continuously- intercommunicated modules: an bladder; and one or more of pressure sensors in connection with said bladder; wearable sensor module 130 comprising one or more of wearable sensors 131, 51 (see FIG. 1, not shown on FIG. 2A; in the embodiment depicted in FIG. 2A, the sleeve also functions as a part of the wearable sensor module 130); “I Feel Good” button 82; and computerized feedbacking module (CFM) 140 comprising a processor and indicator.

Reference is now made to an interactive respiratory relaxation auto-biofeedback device for relieving stress 1000 characterized by at least three continuously-intercommunicated modules: at least one bladder 10 and at least one pressure sensor 52 in connection with the at least one bladder 10; the at least one bladder 10 configured for manual actuation by a user during exhalation; at least one programmable actuator for inflating and deflating the at least one bladder 10; an actuating module, comprising computerized feedbacking module (CFM) 140 for programming and controlling the programmable actuator of the at least one bladder 10, and a self reporting switch 82 for user fixing of the breathing rate set by said actuating module.

Further reference is made to the above-mentioned device 1000 as defined above, further characterized by the device 1000 further comprising at least one wearable sensor 131 for receiving and transmitting breathing data; and a processor for receiving the breathing data, processing the received breathing data, and sending instructions to the actuator for inflating and deflating the at least one bladder 10.

Further reference is made to the above-mentioned device 1000 as defined in any of the above, further characterized by the at least one actuator being selected from the group consisting of: a valve 160, a pump 170, a spring 200, and any combination thereof.

Further reference is made to the above-mentioned device 1000 as defined in any of the above, further characterized by the at least one wearable sensor 131 being configured for sensing regular breathing or diaphragmatic breathing.

Further reference is made to the above-mentioned device 1000 as defined in any of the above, further characterized by the wearable sensor 131 comprising at least one accelerometer configured to measure the movement of a body part to which the at least one wearable sensor 131 is attached.

Further reference is made to the above-mentioned device 1000 as defined in any of the above, further characterized by the at least one wearable sensor 131 having a noise filtering mechanism.

Further reference is made to the above-mentioned device 1000 as defined in any of the above, further characterized by the actuating module being operable in a method comprising steps: by means of the CFM 140, directing a user for breathing and actuating the at least one bladder 10 in a defined rate, namely a rhythm B_{I/D}R to both (i) breathing in a hereto defined rate, namely inhaling/exhaling in the directed rhythm (U_{I/E}R), and concurrently (ii) inflating/deflating the at least one bladder 10 in the hereto defined rhythm (B_{I/D}R); and further concurrently, releasing excess air via the valve from the at least one bladder 10 in a rhythm (U_{P/R}R); and further concurrently, by means of the at least one wearable sensor 131, sensing the user for a breathing rhythm (U_{I/E}R).

Further reference is made to the above-mentioned device 1000 as defined in any of the above, further characterized by user's breathing rhythm U_{I/E}R correlating with the CFM 131 indicating B_{I/D}R, indicating B_{I/D}R until first members of a group consisting following steps being provided: (i) user indicating “I Feel Good” 82; (ii) CFM 140 indicating program is automatically terminated; and (iii) user's breathing rhythm U_{I/E}R not corrollating any longer with the CFM 140 indicated B_{I/D}R, resulting in the CFM 140 indicating end of procedure.

Further reference is made to the above-mentioned device 1000 device as defined in any of the above, further characterized by user's indicating “I Feel Good” 82 and user's UI/ER correlates with BI/DR for a predefined period of time, resulting in the CFM 140 indicating end of procedure.

Further reference is made to the above-mentioned device 1000 as defined in any of the above, further characterized by the programmable actuator comprising a termination program, further characterized by the termination program being activated resulting in the CFM 140 indicating end of procedure.

Further reference is made to the above-mentioned device 1000 as defined in any of the above, further characterized by user's breathing rhythm U_{I/E}R not correlating with the at least one bladder's actuating rhythm B_{I/D}R for more than a predefined period of time, further providing steps being members of a group consisting of (i) indicating the user (e.g., alerting by means of lights, sounds, vibration, reminding by audio or text messages), by means of the CFM 140, to further pressing/releasing the at least one bladder 10 in the indicated rhythm B_{I/D}R; (ii) alerting the user or medical personnel in close or remote location; and (iii) altering rhythm B_{I/D}R.

Further reference is made to the above-mentioned device 1000 as defined in any of the above, further characterized by at least one bladder 10 ibeing passive, in that it does not deflate until a user presses the at least one bladder 10.

Further reference is made to the above-mentioned device 1000 as defined in any of the above, further characterized by the device 1000 being connected to at least one additional device, and being configured to upload or download data to/from the at least one additional device.

Further reference is made to the above-mentioned device 1000 as defined in any of the above, further characterized by the computerized feedback module (CFM) 140 being a mobile device.

Further reference is made to the above-mentioned device 1000 as defined in any of the above, further characterized by the device 1000 comprising a user interface (UI) 62.

Further reference is made to the above-mentioned device 1000 as defined in any of the above, further characterized by the user interface (UI) 62 being a mobile device.

Reference is now being made to an interactive respiratory relaxation auto-biofeedback method for relieving stress characterized by at least three continuously-intercommunicating modules, the method comprising steps of: obtaining at least one bladder 10 and at least one pressure sensor 52 in connection with the at least one bladder 10; the at least one bladder 10 configured for manual actuation by a user during exhalation; and operating the at least one bladder 10; obtaining at least one programmable actuator for inflating and deflating the at least one bladder 10; and operating the at least one programmable actuator; obtaining an actuation module, comprising computerized feedbacking module (CFM) 140 for programming and controlling the actuation of the at least one bladder 10, and a self reporting switch 82 for user fixing of the breathing rate set by the actuating module; and operating the actuation module.

Further reference is being made to the interactive respiratory relaxation auto-biofeedback method as defined above, further characterized by the method further comprising steps of: obtaining at least one wearable sensor 131 for receiving and transmitting breathing data; and operating the at least one wearable sensor 131; obtaining a processor for receiving the breathing data, processing the received breathing data, and sending instructions to the actuator for inflating and deflating the at least one bladder 131; and operating the processor.

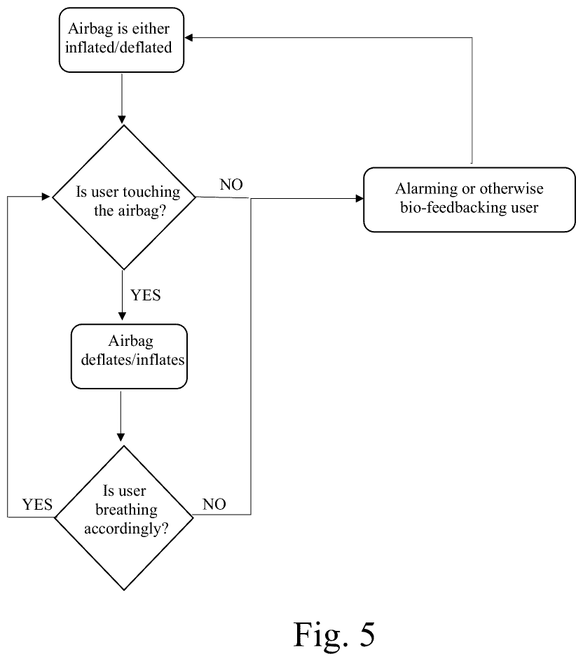

FIG. 5 illustrates the algorithm of operation of CFM 140 in one of the embodiments of the present invention.

Further reference is now made to FIG. 2A, schematically illustrating an embodiment of the invention wherein the device 1000 includes, inter alia, a bladder 10, namely an actuator, bladder, sack, air bag, a flexible envelope filled by liquids (e.g., water-like, oil-like, soap, gel or jelly-like), gas (air, nitrogen etc.) or solids (e.g., micron-sized particulated flowing matter) and a combination thereof, with one or more cells being in fluid connection, with one or more envelopes (all denoted hereto as ‘bladder’).

Further reference is now made to FIG. 2B, schematically illustrating an embodiment of the invention wherein sleeve 150 is made of hard plastic, and the electronic software (electronic integrated micro-controller 60, internal software 61, UX/UI software 62, CFM 140, etc.) is embedded into the plastic sleeve. This embodiment is suitable foe environments where there is a need to sterilize the device 1000 since it is used by multiple users, such as in hospitals.

It is further in scope of some of the embodiments of the invention, wherein the device 100 further comprises a feedback mechanism-thimble which is placed on the user's finger or in the device, when the hand or hands holding the bladder. The thimble-like sensors check stress conditions, like galvanic skin response or other parameters, and translates it to output that appears on the screen 120. The output provided by the device is of various forms: e.g., graph, as a change in light intensity, etc.

[symbol] is further in scope of some of the embodiments of the invention, wherein the pump 170 is operatively connected to the bladder 10 and fills it with air when CAB send such a command. It is further in scope of some of the embodiments of the invention, wherein the bladder 10 is in fluid connection with a pump 170, a vacuum pump, or a release valve 160. Reference is now made to FIGS. 2A, 2B, schematically presenting a release valve 160. When it opens, air escapes from the bladder. The valve 160 is controlled by the controller 110. Pressure sensor 52 measures the air pressure inside the bladder 10.

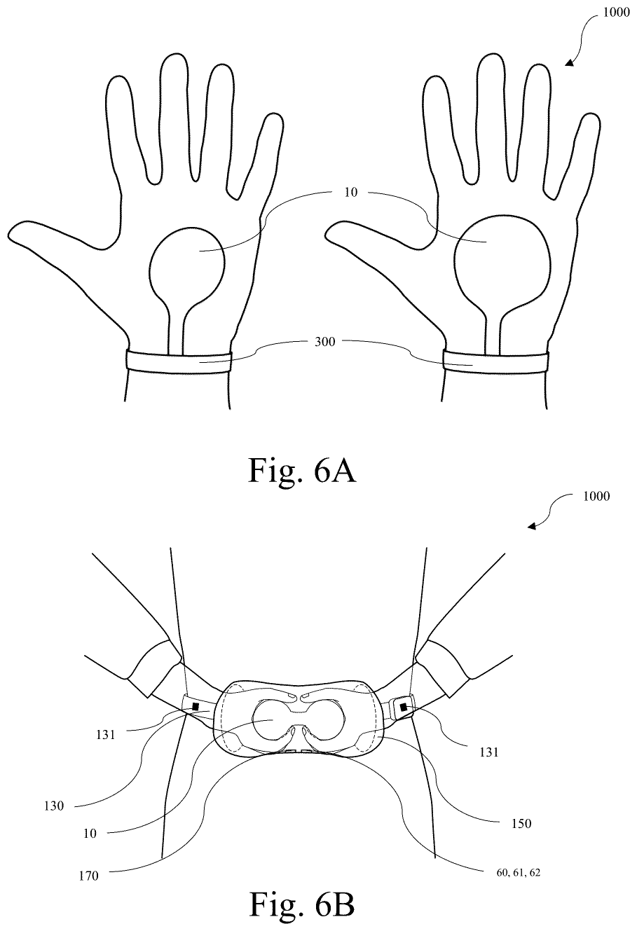

It is further in scope of some of the embodiments of the invention, wherein modules are packed or otherwise enveloped by soft and pleasant-to-touch bundle. A sleeve like configuration is possible, so hands go in the sleeve 150 to hold the bladders 10. A bracelet like configuration 300 is also possible where user holds a bladder and all the other modules are placed inside or embedded in the bracelet. A glove-like and a belt outfit, and roof-like designs and similar configurations are also possible, so hands are positioned under “the roof” 150 or any other article that facilitates the user to hold the bladders in his/her hands and operate the device, see FIG. 6A, FIG. 6B. In the embodiment depicted on FIG. 6B, a belt serve simultaneously as wearable sensor module 130 comprising one or more of wearable sensors 131, and as a holder of “the roof” 150.

Optionally and according to other embodiment of the invention, a linkage bar (not shown) or a strip (such as Velcro™) facilitates a child-user to attach one or more articles he/she loves (that thing gives him a feeling of security and closeness and can help him/her calm down) to the device of the present invention.

According to other embodiment of the invention, CAB controller follows a sequence of steps as hereto defined: Pumping air to the bladder 10 for X seconds, wherein it simulates breathing in (inhaling). The embodiment comprises an overpressure protection—Pressure sensor 52 reads the pressure inside the bladder 10, and in case of overpressure, the controller 110 opens the valve 160, as needed. Stopping the pump 170 for Y seconds, wherein: the valve 160 is opened; starting air-port guides the user that it is time to squeeze the bladder 10 and it simulates breathing out (exhaling). Simultaneously, the reminder 180 (vibration or sound or light) leads the user to squeeze the bladder 10. It facilitates interactive breathing—when the user is squeezing the bladder 10, the pressure inside is increased. The pressure sensor 52 reads the pressure level and accordingly, opens the valve 160. If the user does not squeeze the bladder 10, the valve 160 will stay close and the bladder 10 won't deflate. When the user feels he/she breath at a rate that is pleasant to him/her-he/she clicks the button “I Feel Good” 82 then the inflating/deflating rate stays in the same place and do not decrease.

It is also in the scope of the invention wherein another set of embodiments is provided useful. Hence, a software is useable to monitor the device, and controls the desired sequence. The program includes several steps (or levels or stages), wherein each step has a different cycle time interval and can be repeated according to the user preferences. For example, 11 steps, wherein the breathing rate is slowed-down from step to step. The first step is adapting to the user's own breathing rate. The second step is to reduce to a cycle of about 12 breathings per minute for adults, and about 16 breathings per minute for child. The last step is reaching a cycle of 4 breathes per minute. Each cycle include breathing in and out. Breathing out time interval is longer 1.5-2 than that of the breathing in time interval, in each step. The software has many breathing programs, so that the user can choose which breathing program to do. The software is designed to increase the cycle time as it moves along. User control-the software allows the user to skip, go back, move forward and remain in each step in the sequence, according to his current ability to breathe. Memory—The software can remember in its memory several different sequences, and the user can select from the existing, change or add more sequences.

Along other embodiments of the invention, the controller 110 is preconfigured to start deflating the bladders 10 to thereby inform the user to begin exhaling air, while pressing on the bladders 10 to further deflate them. Air will not flow out of the bladders 10, unless the user applies external pressure onto the bladders 10. If the user does not press the bladders 10, the controller 110 activates a designated reminder unit 180. In a case that a Notification is required, the reminder unit 110 alerts the user to press onto the bladders 10. The alert-reminder may be in a form of audible, visual or vibration performed by the device and/or by a coupled smartphone. If user does not press the bladders 10, the air inside the bladders 10 remain inside the bladders 10, and after a preconfigured time interval, the inhaling cycle begins and the bladder 10 is further inflated. In such a case, an overpressure relief valve 160 will release the excess air from the bladders 10.

According to other embodiment of the invention, the device comprises or otherwise interconnected with additional modules as follows: user has at least one visualization input that indicates the current breathing pace, high breath rate will result a faster visualization, slow breath rate will result slower activity in the visualization.

In case reminder 180 is required, when the user doesn't squeeze the bladder 10 when he/she should (while breathing out), such a reminder 180 may be a buzzer or the like. User holds the bladder 10 and decides which program he/she chooses. The device 1000 operates independently. If the starting rate of the device 1000 is too quick, the user may change the pace so that he can fit in at the selected rate.

The software allows the user to skip/back/forward and remains each step in the sequence, according to his current ability to breathe. From that moment the user continues to breathe with the device 1000, since the respiratory rates become longer (fewer breaths per minute) along the time the user become relaxed.

It is well within the scope and novelty of the device wherein user is guided to integrate three actions at the same time and in parallel:
a. Follow the signaling of the control unit 110 as apparent in the inflate/deflate rate of the bladder/s 10 and triggering of each cycle by the control unit 110 (air is not continued to be released from the release valve if the user does not squeeze to increase pressure);
b. To press/squeeze actively (“doing” action) the bladdern/s 10 with his/her hand/s (preferable two hands) to deflate the bladder/s 10 and once the bladder/s starts to inflate, the user should stop the pressing/squeezing without releasing his grip of the bladder/s 10; and
c. To breath (inhale and exhale) in tandem with the inflate/deflate cycle of the bladder/s 10 and hand/s.

Once bladder 10 starts to inflate the user's grip, the bladder 10 should be relaxed and the user should inhale. Once the bladder 10 begins to deflate, and the reminder 180 is activating, the user should apply pressure to shrink the bladder 10, and the user should exhale. This combined guidance of integrated actions of the user, causes centering of thoughts and focusing on breathing by the user's mind, thus preventing the user's thoughts from wandering away.

Optionally, the device is operatively coupled with a smart-device, having a designated application running thereon. Typically, in such a device, the smart-phone controls operational activities of the device 1000, serving as a control unit 110. The device 1000 and the smart-phone are hence communicate wirelessly, for example, using Bluetooth protocol or similar. The device 1000 receives commands from the dedicated smart-device applications (activating operation, sounds, tag words, graphical display, identification of shift, etc.). The user may program by himself/herself the respiratory rate, can turn on and off 81 the device 1000, can activate a timer, and see a visual indication on the smart-device and/or device display, as pertaining to the inhalation and exhalation instructions, and more. If the user does not press the bladder/s 10, the alert may be in a form of sound, light or vibration by the device and/or by a coupled smart-device. According to a further aspect of the present invention, the focusing on interrelationship of breathing and action allows breathing with the guidelines of the device over time.

With alternative methods based on passive signals (such as light or sound) thoughts have a tendency to wander after a very short time and the intended breathing cycle is interrupted and the user reverts to his original breathing pattern. Intentional slow guided breathing over time makes this relaxation and stress-relief breathing method much more effective than other methods. Even someone who has never practiced relaxation and stress-relief methods or meditation, someone without any experience whatsoever, can benefit from this effective and intuitive device and easily reduce stress. This method is suitable for adults, teens, youth and children. The device can allow a joint session of parent and child with the parent adapting to the breathing cycle of the child. Child and the parent embracing the child are holding together the device. When the bladder inflates and the child is inhaling, the child also feels the swelling of the chest of the parent. They both breathe at the same rate. When the child deflates the bladder together with the parent and exhales, the child also feels the contraction of the chest of the parent. The combination of the device with the activity of the parent allows added affectivity and fast relaxation and stress-relief over time. Simultaneously induced group breathing: the embodiments of the hardware/software-controlled device and the connection of the device to other devices via internet or similar communications media allow multiple devices, not limited in number, in separate physical locations as well a single group location, to be synchronized by a master/slave configuration. A maximum time delay of not more than 50 msec can be utilized even for a global synchronization group of users not limited in number. This supports applications such as controlled synchronized breathing for sports training (eg archery) and such as for group synchronized meditation sessions. The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the following claims.

According to yet other embodiments of the invention, the aforesaid device does not comprise a bladder with a pump, but utilizes a spring inside, see FIG. 2C. The spring 200 is characterized by spring force 210, which counteracts the force 220 applied by the user. When check valve 161 is opened, air flow 230 enters the bladder, and when check valve 162 is opened, air flow 230 exits the bladder.

Alternatively, the bladder 10 is a sponge with a memory and with a spring inside. User is guided to press a spring while exhaling and a sponge returns to its preliminary status and shape during inhaling.

Additional embodiments without a pump are presented in figures FIG. 3B and FIG. 3C.

According to yet other embodiments of the invention, the aforesaid device works in a three steps procedure as follows:
Follow The bladder inflates and deflates;
Do Squeeze the bladder to deflate (and release as it starts to inflate); and
Breath Synchronize your belly breath with the bladder's action.

Reference is made to FIG. 2A and 2B. User holds two bladders 10 inside a protective sleeve 150 that inflates and deflates following device's software. User synchronizes and matches his/her breathing to the rate of bladder(s) 10 inflation/deflation frequency or rate (hereto “rhythm”), which decreases over time. Inhaling and exhaling at a slow rate, activates the brain's para-sympathetic device to induce relaxation of the body. User's hand activity in pressing or squeezing the bladder(s) 10, leads to a continuous and constant interaction between the user and the device 1000, keeping the mind from wandering. Moreover, relaxing emphasizes abdominal breathing and thus increases the amount of air entering the lungs to prevent the breath from staying shallow.

It is according to other embodiments of the invention wherein the device and device of the present invention comprises Electronic circuit, Air pump, Discharge valve for air, Air passages and piping, circuit breaker, On/off indicator, Power Supply-e.g., 12V, air bladder duct, balloon-like bladder, Decorative hollow pillow, Data collection sensors (such as pulse, HRV, oxygen rotation, etc.), Bluetooth connection to connect various modules and remote locations, A mode selector switch will enable basic control (child, adult, sport, labor, diving etc.), A screen, Software that instructs bladders to swell or take out air. The bladders take out air only if the user clicks on them. The bladders have mechanisms that allow the air to come out only by pressure. (Whether by a spring in the bladders or by a valve), blower, pressure valve, The device inflates the bladder at preset times and speeds.

A PC or smartphone interface is configured to provided one or more of the following: Allows the device to turn on and off (in the same way as the button on the box), Allows user to save presets (such as Child, Adult, Other). Each stage is defined by one or more of the following: Inhale time, Exhale time, Breathing time, Standby time between iterations, Number of iterations to combine, The pace column: (NSC) is calculated from the above data by the software.

As said above, a REMINDER 180 is configured to emit an alarm, e.g., a vibration or sound that reminds the user to click on the bladder 10 in case it is distracted. “I Feel Good” button 82, when user feels relaxed, he/she clicks a button and leaves the pace at which he/she feels good (meaning the software will stop the bladders' 10 inflating/deflating rate go down and leave it at the same rate of the rest of the session. For example: If user feels good at 7 breath per minute, this will stay until he/she stops the device).

Squeeze valve 160 will force the user to squeeze the bladder 10 in order to exhale with the squeezing.

Several other examples will be provided showing variants of the device 1000 comprising shells or wings 10. (the next pages are pretty much redundant)

This rating device is a non-intrusive way to help the manufacturer, the physician and the user to monitor the “positive” impact of the use of DABA on user's quality of life (stress relief, emotional control, performance-oriented goals, etc.).

Unlike existing devices that tell the user that he is supposed to feel better, with DABA, the user inputs a signal using the button provided 80, to indicate his/her state of mind.

Following the user previous approval, DABA enable the software to monitor behavioral feedback:
how much time the user is practicing breathing exercises;
the time, date and duration of each session;
if the user is executing the exercise within the chosen mode parameters (too slow, too fast, too tense, etc.)

Based on the data provided by the user, the software can provide tips and advice to the user.

Each of the modes have different breathing patterns, their respective timing are adapted to the level of efficiency and complexity the user is looking for.

The user can also choose the mode he/she wants directly from the device, without needing the external device.

Using the software 60, 61, 62, the user can choose the frequency of the breathing sessions and set reminders 180 via messaging or notifications and/or the DABA itself. In this last case, DABA can emit different kind of signals: vibrations, lights and movements.

If the user wishes to do so, the user can agree to share his/her geographic location (internet IP, smartphone GPS, etc.) to receive information about DABA users close by, breathing experts and/or service providers around him/her, join a group breathing session, and more.

In another embodiment, one DABA can be used to remotely control another DABA, using the wireless adaptor 140, in the same room and/or without geographic limitation via an internet connection. It can also be used to interactively guiding or sharing a breathing session (for relaxation, stress relief or performance purposes).

In another embodiment two or more DABAs can be synchronized to share the same breathing pattern at the same time using the software, in the same room and/or without geographic limitation via an internet and/or wireless connection.

Simultaneously induced group breathing: The embodiments of the hardware/software-controlled device and the connection of the device to other devices via internet or similar communications media allow multiple devices, not limited in number, in separate physical locations as well a single group location, to be synchronized by a master/slave configuration. A maximum time delay of not more than 50 milliseconds can be utilized even for a global synchronization group of users, not limited in number. This supports applications such as controlled synchronized breathing for sports training and such as for group synchronized meditation sessions.

It is another intention of the present invention to enable procedural memory and muscle memory effect, increasing the efficiency of learning and practicing breathing patterns (e.g. singing).

It is in the scope of the invention wherein an interactive focus respiratory relaxation and stress-relief device 1000 is disclosed. The device comprises at least one adjustable member 10 operatively connected to a motor 30 to actuate adjustment of said member 10; wherein said at least one adjustable member 10 is configured for at least one first state and one at least second state, the motor 30 configured for actuating the at least one adjustable member 10 to move reciprocally from the at least one first state to the at least one second state according to a predetermined protocol.

It is also in the scope of the invention wherein the device 1000 defined above further comprises a power source 20 for operating the motor 30, e.g., rechargeable batteries.

It is also in the scope of the invention wherein the device 1000 in any of defined above comprises at least one operative button 80.

It is also in the scope of the invention wherein the device 1000 in any of defined above comprises at least one operative button 80, operates the actions selected from a group consisting of: a. turning on the device; b. maintaining the speed of the device; c. controlling the speed of the device; d. stopping the action of the device; e. activating feedback mode to the device; f. selecting operating modes of the device; g. start session, and any combination thereof.

It is also in the scope of the invention wherein the device in any of defined above comprises at least one LED 90 for providing visual information to the user.

It is also in the scope of the invention wherein the device 1000 in any of defined above comprises at least one pressure sensor 50 operatively connected to the member 10 for detecting parameter signals from the user.

It is also in the scope of the invention wherein the device 1000 in any of defined above comprises at least one wearable sensor 131.

It is also in the scope of the invention wherein the device 1000 in any of defined above comprises at least one electronic integrated micro-controller 110 having a CRM 70 comprising instructions for operating the motor 30 in a predetermined manner.

It is also in the scope of the invention wherein the device 1000 in any of defined above comprises CRM 70 that provides data storage concerned with operating the device 1000 and the data collected from the device 1000.

It is also in the scope of the invention wherein the device 1000 in any of defined above comprises at least one wireless adaptor 40 interconnected to the at least one electronic integrated micro-controller 110 for connecting the device 1000 with external devices.

It is also in the scope of the invention wherein the device 1000 in any of defined above comprises comprises at least one optional screen or touch screen 120 for interacting with the device 1000.

It is also in the scope of the invention wherein the device 1000 in any of defined above comprises or interconnected with at least one database 100 via said at least one wireless adaptor 40; the database is allocated in at least one data server 100.

It is also in the scope of the invention wherein the device 1000 in any of defined above comprises of interconnected to an external device via the at least one wireless adaptor 40.

It is also in the scope of the invention wherein the device 1000 in any of defined above comprises external device selected from a group consisting of smartphones, personal computers, laptops, tablets, smart watches and any combination thereof.

It is also in the scope of the invention wherein the device 1000 in any of defined above comprises an external device which comprises UX/UI software 62.

It is also in the scope of the invention wherein the UX/UI software 62 enables the user to use the device 1000 and to access the database 100. It is also in the scope of the invention wherein the UX/UI software 62 is compatible with all conventional Operating Devices (OS).

It is also in the scope of the invention wherein the device 1000 in any of defined above is a wearable device 130.

It is also in the scope of the invention wherein the at least one electronic integrated micro-controller 110 further comprises internal software 61 that enables synchronization with the external device, enables control of the motor 30 and the at least one LED 90 and enables collection of data from the at least one sensor 50, 131.

It is also in the scope of the invention to discloses a method of interactive respiratory relaxation and stress-relief comprising the steps of acquiring an interactive respiratory relaxation and stress-relief device 1000, the device comprising an adjustable member 10 operatively connected to a motor 30 to actuate adjustment of the member 10, the at least one adjustable member 10 is configured for at least one first state and one at least second state; turning on the device; choosing one mode from at least one modes; and pressing start button 80; wherein the method further comprises a step of synchronizing respiratory movement and body movements with the movements of the device 1000.

1-45. (canceled)

46. An interactive respiratory relaxation auto-biofeedback device for relieving stress, said device characterized by at least five continuously-intercommunicated modules:
a) at least one bladder and at least one pressure sensor in connection with said at least one bladder; said at least one bladder embedded into an article configured to facilitate a user to hold said at least one bladder;
b) at least one programmable actuator for inflating and deflating said at least one bladder;
c) an actuating module, comprising a computerized module for programming and controlling said programmable actuator of said at least one bladder, and a self reporting switch for the user fixing of the breathing rate set by said actuating module;
d) at least one controller unit for activating said programmable actuator; said at least one controller unit being an external controller unit or an integrated controller unit;
e) a reminder unit configured to lead the user to actively deflate said at least one bladder; wherein said at least one bladder is configured for active manual actuation by the user during exhalation by means of said at least one bladder not deflating automatically unless one or more of the following occurs: i. said at least one pressure sensor detects overpressure in said at least one bladder, concurrently activating said controller unit, said controller unit concurrently activating said programmable actuator; ii. said controller unit preconfigured to start deflating said at least one bladder to thereby inform the user to begin simultaneously exhaling air and squeeze said at least one bladder to further deflate said at least one bladder.

47. The device of claim 46, wherein said article comprises at least one of the following:
a sleeve-like configuration; a bracelet-like configuration; a glove-like configuration; and a roof-like configuration; further wherein said article is packed or otherwise enveloped by a soft and pleasant-to-touch bundle.

48. The device of claim 46, wherein said device further comprises:
a) at least one wearable sensor module comprising at least one sensor for receiving and transmitting breathing data; and
b) a processor for receiving said breathing data, processing said received breathing data, and sending instructions to said actuator for inflating and deflating said at least one bladder.

49. The device of claim 48, wherein said wearable sensor module is a belt outfit attached to said article.

50. The device of claim 46, wherein at least one of the following is true:
a) said at least one actuator is selected from the group consisting of: a valve, a pump, a spring, and any combination thereof; and
b) said reminder unit leads the user to squeeze said at least one bladder by at least one of the following means: vibration, sound, light, buzzer, audio message, text message.

51. The device of claim 46, wherein said programmable actuator actuator is programmed to perform the following two step cyclic algorithm: i. to inflate said at least one bladder for X seconds to facilitate the user to inhale; and further concurrently, ii. to stop inflating said at least one bladder for Y seconds to facilitate the user to exhale; further wherein the value of said Y is 1.5-2 times larger than the value of said X.

52. The device of claim 46, wherein said at least one bladder is passive, in that it does not deflate until a user squeezes said at least one bladder.

53. The device of claim 46, further characterized by said device being connected to at least one additional device, and being configured to upload or download data to/from said at least one additional device.

54. The device of claim 46, further comprising a set of at least one ear phone that connects to said device, and enable the user to listen to music or rhythms, wherein said music or rhythms are automatically synchronized with the user's breathing rate via said controller unit.

55. An interactive respiratory relaxation auto-biofeedback method for relieving stress, said method comprising steps of:
a) obtaining an interactive respiratory relaxation auto-biofeedback device for relieving stress, said device characterized by at least five continuously-intercommunicated modules: i) at least one bladder and at least one pressure sensor in connection with said at least one bladder; said at least one bladder embedded into an article configured to facilitate a user to hold said at least one bladder; ii) at least one programmable actuator for inflating and deflating said at least one bladder; iii) an actuation module, comprising computerized module for programming and controlling said actuation of said at least one bladder, and a self reporting switch for the user fixing of the breathing rate set by said actuating module; iv) at least one controller unit for activating said programmable actuator; said at least one controller unit being an external controller unit or an integrated controller unit; v) a reminder unit configured to lead the user to actively deflate said at least one bladder; wherein said at least one bladder is configured for active manual actuation by the user during exhalation by means of said at least one bladder not deflating automatically unless one or more of the following occurs: i) said at least one pressure sensor detects overpressure in said at least one bladder, concurrently activating said controller unit, said controller unit concurrently activating said programmable actuator; ii) said controller unit preconfigured to start deflating said at least one bladder to thereby inform the user to begin simultaneously exhaling air and squeeze said at least one bladder to further deflate said at least one bladder; and
b) steps of operating said device by a user, comprising cyclic algorithm of steps of said user keeping his/her hands inside said article and continuously touching said at least one bladder and following the changes of size of said at least one bladder, said user simultaneously inhaling, while said at least one bladder inflates; and concurrently, said user simultaneously both exhaling and squeezing said at least one bladder causing said at least one bladder to deflate, while said at least one bladder does not inflate.

56. The method of claim 55, wherein said method further comprises steps of:
a) obtaining said device, said device further comprising: i. at least one wearable sensor module comprising at least one sensor for receiving and transmitting breathing data; ii. a processor for receiving said breathing data, processing said received breathing data, and sending instructions to said actuator for inflating and deflating said at least one bladder; and
b) operating said device.

57. The method of claim 56, wherein said at least one wearable sensor is configured for sensing regular breathing or diaphragmatic breathing; the steps of operating said device by the user, further comprising cyclic algorithm of steps of said user inflating his/her stomach simultaneously with keeping his/her hands inside said article, continuously touching said at least one bladder, and inhaling, while said at least one bladder inflates; and concurrently, simultaneously deflating his/her stomach, exhaling and squeezing said at least one bladder causing said at least one bladder to deflate, while said at least one bladder does not inflate.

58. The method of claim 55, wherein said steps of operating said actuation module comprise steps of saving preset operation regimes on said computerized module, said preset operation regimes comprise at least one of: child, adult, sport, labor, diving, other, and any combination thereof.

59. The method of claim 55, wherein said steps of operating said actuation module comprise steps of programming said computerized module to define each stage of operation of said at least one programmable actuator.

60. The method of claim 59, wherein said each stage of operation of said at least one programmable actuator is defined by at least one of the following: inhale time, exhale time, breathing time, standby time between iterations, and number of iterations to combine; and wherein inflating/deflating pace column is calculated from inhale time, exhale time, breathing time, standby time between iterations, and number of iterations to combine.

61. The method of claim 60, wherein each one of said each stage of operation has a different cycle time interval and can be repeated according to the user preferences.

62. The method of claim 61, wherein said inflating/deflating pace is configured to decrease from a preceding stage of operation to a following stage of operation; further wherein when the user feels relaxed, said user indicates “I Feel Good” by clicking said self reporting switch, and stops said inflating/deflating pace from further decreasing.

63. The method of claim 55, said user being a child assisted by a parent, wherein said method further comprise steps of said parent adapting to breathing cycle of said child, said parent embracing said child, and said child feeling swelling and concurrent contraction of said parent's chest.

64. The method of claim 55, wherein said method further comprises steps of: obtaining at least one additional device; and operating said device and said at least one additional device by connecting said devise to said at least one additional device, said being configured to upload or download data to/from said at least one additional device.

65. The method of claim 64, further comprising steps of simultaneously induced group breathing, said steps of simultaneously induced group breathing comprising steps of obtaining internet connection or other communication media; connecting said device and said at least one additional device to internet connection or other communication media; and operating said device, said at least one additional device, and said internet connection or other communication media to synchronize the activity of said device and said at least one additional device in master/slave configuration.
